(19) United States
(12) Patent Application Publication
(10) Pub. No.: US 2011/0117175 A1
(43) Pub. Date: May 19, 2011

(54) SWEET ANALGESIC FOR USE IN MEDICAL PROCEDURES OR TREATMENTS

(76) Inventor: Richard J. Rosenbaum, Highland Ranch, CO (US)

(21) Appl. No.: 12/874,038

(22) Filed: Sep. 1, 2010

Related U.S. Application Data
(60) Provisional application No. 61/262,433, filed on Nov. 18, 2009.

(51) Int. Cl. A61K 9/70; A61K 31/7016; A61P 29/00

(52) U.S. Cl. 424/443; 514/53

(57) ABSTRACT

Sweet analgesics and compositions comprising a sweet analgesic and a delivery vehicle for reducing pain or discomfort associated with a medical procedure or treatment are disclosed. The sweet analgesic is administered or delivered to the intra-oral cavity of the patient prior to or during the medical procedure or treatment to increase the comfort of the patient. The sweet analgesic can be applied to a dental roll or pad and the roll or pad can be inserted between the cheek and gum of the patient to allow for the release of the sweet analgesic during the medical procedure or treatment. The dental roll or pad can be replaced as necessary to maintain the release of the sweet analgesic into the intra-oral cavity of the patient during the medical procedure or treatment.

### FIELD OF THE INVENTION

The present invention relates generally to a pain reliever for medical procedures or medical treatments comprising a sweet analgesic and a delivery vehicle for oral delivery of the sweet analgesic. More particularly, the invention relates to the application of a natural sweetener, artificial sweetener, or combination thereof to the oral cavity of a patient prior to and during a medical procedure or treatment. More particularly still, an apparatus, kit and method of the present invention includes a delivery vehicle comprising a suitable amount of natural sweetener, artificial sweetener, or combination thereof for insertion into the oral cavity of a patient to relieve pain during medical procedures or treatments.

### BACKGROUND

Research has shown that the administration of sucrose to an infant or neonate prior to a painful procedure decreases the pain response. The administration of sugar is believed to release brain endorphins, which in turn moderate the infants' or neonates' response to the pain. Some procedures where sugar has been administered to infants and neonates include blood draws, the placing of IVs, circumcisions, and immunizations. The sugar is typically administered by using an ampoule to place 1 to 2 drops directly on to the infants' or neonates' tongue or pacifier. The infant or neonate response is quick, typically within 10 seconds and lasts for up to 10 minutes.

Research suggests the effects of sucrose analgesics are dependent upon oral administration. Lefrak et al. Sucrose Analgesia: Identifying Potentially Better Practices. Pediatrics. 118:2 (2006). The release of brain endorphins associated with the sweet taste of the sucrose is believed to promote the decrease in pain response. Surprisingly, using sucrose to relieve pain in patients older than infants has been unsuccessful. Pepino et al. Sucrose-Induced Analgesia is related to Sweet Preferences in Children but not Adults. Pain. December 15; 119(1-3): 210-213 (2005). The results from recent studies find the use of sucrose as an analgesic is generally limited to infants and neonates. While the exact reasons are not understood, the administration of sucrose analgesic to patients older than infants is not believed to decrease the pain response.

The use of a sweet analgesic has several advantages. A sweet analgesic is easy to administer and convenient to use. A sweet analgesic does not require the use of needles and the administration may be supplemented as necessary to meet the patients' needs. The sweet taste is generally agreeable with most patients, and studies show no increase in blood glucose levels with the minimal amount of sugar used. Therefore, a sweet analgesic suitable for use in pediatric, adolescent, and/or adult patients is desirable.

### SUMMARY OF THE INVENTION

The present invention increases patient comfort of a pediatric, adolescent, or adult patient undergoing a medical procedure or treatment. As used herein, medical procedure or treatments comprises dental procedures or treatments. A product according to the present invention allows the delivery of a sweet analgesic to the intra-oral cavity of an adolescent or adult patient immediately prior to and during the procedure or treatment. Examples of medical procedures and treatments include but are not limited to intra-oral procedures and physical therapy treatments. The sweet analgesic may be sucrose and water, an artificial sweetener and water, or a combination of sweeteners, water, and other pharmaceutically acceptable ingredients. The sweet analgesic may be delivered using a common dental grade cotton gauze roll or pad that has been presoaked with the sweet analgesic. The delivery of the sweet analgesic can also be accomplished using capsule, pill, tablet, orally disintegrating tablet, film, elixir, emulsion, syrup, gel, gum, suspension or tincture.

A method according to the present invention includes using the delivery vehicle to place the sweet analgesic into the intra-oral cavity of a child, adolescent, or adult patient prior to and/or during a medical procedure or treatment. The sweet analgesic can be placed directly on the patient's tongue, placed under the tongue, or between the lip or check and gum using a suitable delivery vehicle.

A kit according to the present invention includes multiple prepackaged doses of sweet analgesic with accompanying delivery vehicle and instructions. The kit can include prepackaged and sterilized single dose dental grade cotton rolls or pads, capsules, pills, tablets, orally disintegrating tablets, film, elixir, emulsion, syrup, suspension or tincture comprising a suitable amount of the sweet analgesic for reducing pain in the patient. The kit can include sweet analgesic in a range of different concentrations to benefit a variety of pediatric, adolescent, or adult patients.

### BRIEF DESCRIPTION OF THE DRAWINGS

FIG. 1 shows patient responses to the use of a sweetened dental roll of the invention during a dental cleaning. 16 total patients were tested. Ten patients (Group 1) experienced a better comfort level as a result of using the sweetened dental roll during the dental cleaning. Five patients (Group 2) did not experience any change in comfort level as a result of using the sweetened dental roll. Only one patient (Group 3) experienced a worse comfort level as a result of using the sweetened dental roll.

FIG. 2 shows the degree to which the sweet analgesic was capable of improving patient comfort. Of the ten patients who reported a better comfort level, one patient (C) described the experience as much better, three patients (B) described the experience as better, and six patients (A) described the experience as slightly better.

### DETAILED DESCRIPTION OF THE INVENTION

While the invention will be described with respect to preferred embodiment configurations and with respect to particular devices used therein, it will be understood that the invention is not to be construed as limited in any manner by either such configuration or components described herein. Also, while particular delivery vehicles and analgesics are described herein, it will be understood that such particular colloids and administration routes are not to be construed in a limiting manner. Instead, the principles of this invention extend to any manner of applying sweet analgesics to the intra-oral cavity of a patient to reduce the pain response associated with a medical procedure or treatment. As used herein, medical procedure or treatment comprises dental procedures or treatments, physical therapy procedures or treatments, and the treatment of chronic pain. These and other variations of the invention will become apparent to those skilled in the art upon a more detailed description of the invention.

The advantages and features which characterize the invention are pointed out with particularity in the claims annexed hereto and forming a part hereof. For a better understanding of the invention, however, reference should be had to the accompanying descriptive matter, in which there is illustrated and described a preferred embodiment of the invention. Thus, one of skill in the art will appreciate that the present invention transfers to other environments and applications.

Prevailing theory suggests the use of sucrose as an analgesic is limited to infants and neonates, and recent studies of adult woman have shown that the use of sucrose as an analgesic is ineffective in adult patients. Surprisingly and in direct contrast to recent studies and the prevailing theory, the present invention demonstrates the use of a sweet analgesic to increase the comfort of non-infant or neonate patients, such as adult patients, during medical procedures or treatments. The present invention is directed to a product and methods for relieving pain and increasing the comfort of patients during medical procedures or medical treatments. Without being limited to any particular theory, the present invention may be effective due to the continuous administration of sweet analgesic and/or the change in sweetness level.

The patients can be pediatric, adolescent, or adult patients. In a preferred embodiment, the patients are adult patients. Examples of medical procedures and treatments include but are not limited to intra-oral dental procedures, physical therapy treatments, blood drawing, IV administration, administration of local anesthetics, cryotherapy, stapling, suturing, immunization, and chronic pain. Examples of chronic pain include but are not limited to arthritis, back pain, headaches, spinal stenosis, sciatica neuritis, fibromyalgia, complex regional pain syndrome, cancer pain and psychogenic pain. In a preferred embodiment, the medical procedure or treatment comprises an intra-oral procedure. Examples of intra-oral procedures include but are not limited to dental cleaning, fillings, tooth extractions, root canals, bleaching, the addition or removal of sealant, the addition or removal of orthodontics, the addition or removal of dentures, dental caps, or dental bridges.

The sweet analgesic of the invention is easy to administer and convenient to use, does not require the use of needles, may be supplemented as necessary to meet a patient's needs, and comprises a sweet taste that is generally agreeable with most patients. The composition of the invention delivers a sweet analgesic to the intra-oral cavity of the patient using a delivery vehicle prior to and during the medical procedure or treatment. A composition of the present invention comprises a sweet analgesic and a delivery vehicle for delivering the sweet analgesic to the intra-oral cavity of a patient. The sweet analgesic can be naturally occurring or artificial. Examples of suitable sweeteners include but are not limited to sucrose, glucose, fructose, dextrose, maltodextrin, corn syrup, high fructose corn syrup, cyclamate, aspartame, sucralose, xylitol, cyclamate, stevia, brazzein, curculin, erythritol, glycyrrhizin, honey, luo han gua, mabinlin, monatin, miraculin, monellin, pentadin, thaumatin, acesulfame potassium, alitame, salt of aspartame-acesulfame, dulcin, glucin, neohesperidin dihydrochalcone, neotame, P-4000, saccharin, or a combination thereof. The sweet analgesic of the invention can be a mixture of one or more sweeteners that are natural, artificial or a combination thereof.

The sweet analgesic can be mixed with water or with a variety of other pharmaceutically acceptable ingredients. For example, the sweet analgesic can include the addition of thickening agents, preservatives, antibiotics, additional analgesics, coloring agents, tabletting agents or any number of additional pharmaceutically acceptable agents and combinations thereof. In one embodiment, the sweet analgesic is sucralose in water. In another embodiment, the sweet analgesic is aspartame and sucrose in water with the addition of thickening agents to produce a gel.

The sweet analgesic can be a solid, gel, solution, or semisolid. The sweet analgesic can include a compound, molecule, or treatment that elicits a biological response. For example, a therapeutic agent, pharmaceutical agent, a vaccine, an immunological agent, a local or generic anesthetic or painkiller, such as articaine, novicaine, lidocaine, acetaminophen, demerol, aspirin, ibuprofen, an antigen, or a protein or peptide and antibodies.

The delivery vehicle for the sweet analgesic can be a solid, gel, solution, or semisolid. For example the delivery vehicle for the sweet analgesic can be a capsule, pill, tablet, dental roll or pad, gauze, film, elixir, gel, gum, emulsion, syrup, ampoule, suspension, tincture, or combination thereof. In an embodiment, a composition of the invention comprises a sweet analgesic solution and an ampoule as the delivery vehicle for the sweet analgesic solution. In another embodiment, the delivery vehicle comprises a dental grade roll or pad soaked with a solution comprising a suitable amount of sweet analgesic for reducing pain in a pediatric, adolescent, or adult patient. In yet another embodiment, the delivery vehicles comprises a sweet analgesic in the form of a gel. The delivery vehicle can be selected to result in an immediate release of the sweet analgesic or to provide a continuous or time delayed release of the sweet analgesic.

In some embodiments, the delivery vehicle will permit the extended release or delayed release of the sweet analgesic. Extended release or delayed release methods are well know in the art and include the use of capsules, pills, gels and tablets that dissolve slowly and release the sweet analgesic over time. Examples include, but are not limited to, tablets with an inert porous matrix or capsules comprising granules with a retarding coating or a disintegrating coating.

In an embodiment, tablets with an inert porous matrix may be obtained by mixing the sweet analgesic with water insoluble polymer or waxes, filler, and binders. An example of suitable diffusion-retarding polymers includes polyvinylacetate, polyvinylchloride, ethylcellulose and paraffin. The fillers and binders may be solid, powdered carriers such as lactose, saccharose, sorbitol, mannitol, starch, amylopectin, cellulose derivative, gelatin or other suitable carrier. The mixture is moistened with a solvent, for example, water or ethanol or a solution comprising water and a polymer. The solution may be formed into a tablet of suitable size and shape. In some embodiments, the tablet may contain a lubricating agent, for example, magnesium stearate, calcium stearate, sodium stearyl fumarate or polyethyleneglycol wax.

In another embodiment, capsules comprising granules with extended release characteristics are obtained by making a core material comprising the sweet analgesic and fillers. The surface of the core material is then coated with diffusion-retarding water insoluble polymers or waxes. The resulting granules are then placed into gelatin capsules. The core material could be prepared by mixing the sweet analgesic with carefully selected fillers such as lactose, sorbitol, starch, cellulose derivatives or other suitable fillers. The mixture is moistened with a solvent, for example, water or ethanol or a solution consisting of water and a polymer. The mass is formed into granules, for example, by extrusion and spheronization. The surfaces of the granules are coated with a solution consisting of a solvent, e.g., methylene chloride and/or isopropyl alcohol, and water insoluble polymers, e.g., ethylcellulose. The granules are then filled into gelatin capsules. There are also other types of extended release preparations that may be suitable for use with a sweet analgesic that are well known in the art and will be apparent to a skilled artisan.

The sweet analgesic is generally administered within the patient's oral cavity. Placement within the oral cavity can be dependent on the delivery vehicle. For example, a sweet analgesic delivered in solution from an ampoule can be placed directly on the tongue, under the tongue or between the patient's lip or cheek and gum. A sweet analgesic delivered, for example, by dental grade roll or pad, tablet, or capsule and the like can be placed under the tongue or between the patient's lip or cheek and gum. Although other delivery vehicles are suitable for such delivery of the sweet analgesic and can be placed under the tongue or between the patient's lip or cheek and gum. In some embodiments, the patient will be able to suck, milk, or agitate the sweet analgesic to stimulate further release. The positioning of the sweet analgesic is also dependant upon the type or location of the medical procedure or treatment. For example, in intra-oral procedures, if the right upper quadrant of a patient's mouth is the working area, the sweet analgesic may be placed in the lower left quadrant.

The concentration of the sweet analgesic is described herein with reference to sucralose as a sweetness standard. Sweetening equivalent amount as used herein means the amount of a sweetener or combination thereof to achieve an equivalent sweetness to a reference amount of sucralose. For example, sucralose is 600 times sweeter than sucrose, resulting in an equivalent concentration of sucralose that is 600 times less that the concentration of sucrose to achieve the same level of sweetness. For example, Table 1 compares the sweetness of some common low calorie sweeteners to sucrose.

TABLE 1

| | Acesulfame | | Rebiana | | |
|---|---|---|---|---|---|
| | Potassium | Aspartame | (rebaudioside A) | Saccharin | Sucralose |
| Common | Sweet One ® | NutraSweet ® | Truvia ™ | Sweet N | Splenda ® |
| Brand Name | | Equal ® | | Low ® | |
| Sweetness | ≈200 times | ≈200 times | ≈200 times | ≈300 times | ≈600 times |
| Relative to | sweeter | sweeter | sweeter | sweeter | sweeter |
| Sucrose | | | | | |

In an embodiment, the sweet analgesic comprises about 0.00002 to about 0.5 grams, about 0.00002 to about 0.4 grams, about 0.00002 to about 0.3 grams, about 0.00002 to about 0.002 grams, about 0.00002 to about 0.1 grams, about 0.00002 to about 0.09 grams, about 0.00002 to about 0.08 grams, about 0.00002 to about 0.07 grams, about 0.00002 to about 0.06 grams, or about 0.00002 to about 0.05 grams sucralose or a sweetening equivalent amount thereof. In another embodiment, the sweet analgesic comprises about 0.015 to about 0.2 grams, about 0.015 to about 0.1 grams, about 0.015 to about 0.09 grams, about 0.015 to about 0.08 grams, about 0.015 to about 0.07 grams, or about 0.015 to about 0.06 gram sucralose or a sweetening equivalent amount thereof. In another embodiment, the sweet analgesic comprises about 0.001 to about 0.2 grams, about 0.001 to about 0.1 grams, about 0.001 to about 0.09 grams, about 0.001 to about 0.08 grams, about 0.001 to about 0.07 grams, or about 0.001 to about 0.06 gram sucralose or a sweetening equivalent amount thereof. In yet another embodiment, the sweet analgesic comprises about 0.000001, 0.00001, 0.00002, 0.00003, 0.00004, 0.00005, 0.00006, 0.00007, 0.00008, 0.00009, 0.0001, 0.0002, 0.0003, 0.0004, 0.0005, 0.0006, 0.0007, 0.0008, 0.0009, 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.10, 0.11, 0.12, 0.13, 0.14, 0.15, 0.16, 0.17, 0.18, 0.19, 0.2, 0.21, 0.22, 0.23, 0.24, 0.25, 0.26, 0.27, 0.28, 0.29, 0.3, 0.31, 0.32, 0.33, 0.34, 0.35, 0.36, 0.37, 0.38, 0.39, 0.4, 0.41, 0.42, 0.43, 0.44, 0.45, 0.46, 0.47, 0.48, 0.49, or 0.5 grams sucralose or a sweetening equivalent amount thereof.

In other embodiments, for example, the sweet analgesic comprises about 0.0001 grams to about 5.0 grams, about 0.0001 grams to about 5.0 grams, about 0.001 grams to about 5.0 grams, about 0.01 grams to about 5.0 grams, about 0.1 grams to about 5.0 grams, or about 1 gram to about 5.0 grams. In another embodiment, the sweet analgesic comprises about 0.001 grams to about 2 grams, 0.01 grams to about 2 grams, about 0.02 grams to about 1.9 grams, about 0.03 grams to about 1.8 grams, about 0.04 grams to about 1.7 grams, about 0.05 to about 1.5 grams, about 0.5 grams to about 1.0 grams, 1.0 grams to about 1.5 grams, about 1.1 grams to about 1.4 grams, or about 1.2 grams to about 1.3 grams. In yet another embodiment, the sweet analgesic comprises about 0.1. 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, or 5.0 grams sucralose or a sweetening equivalent amount thereof.

The concentration of the sweet analgesic can be dependent upon the method of delivery. When the sweet analgesic is delivered directly to the tongue, for example, as a solution by ampoule, the concentration may be higher or lower compared to when the sweet analgesic is delivered, for example, in a dental roll or pad. The concentration of the sweet analgesic can also change relative to the patient's preferences and sensitivity to sweetness. One patient may prefer and notice an increased comfort level with a composition comprising more or less sweet analgesic compared to another patient.

A method of the present invention comprises administering an amount of sweet analgesic to a patient to increase the patient's comfort level and decrease the patient's pain response associated with a medical procedure or treatment. The medical procedure or treatment includes but is not limited to intra-oral procedures and physical therapy treatments. In an embodiment, the procedure is an intra-oral procedure. Examples of intra-oral procedures include but are not limited to fillings, root canals, teeth cleaning, extractions, bleaching, the addition or removal of sealants, the attachment or removal of braces or other orthodontics, the addition or removal of dentures, caps, and/or dental bridges. The patient can be a pediatric, adolescent, or adult patient. In an embodiment, the patient is an adult patient. The sweet analgesic of the invention is generally administered to the patient by placing a sweet analgesic or composition of the invention into the intra-oral cavity of a patient. The sweet analgesic can be administered to the patient's intra-oral cavity immediately prior to the procedure or minutes before the procedure. In some embodiments, the sweet analgesic is administered to the patient's intra-oral cavity within seconds of the procedure beginning. In other embodiments, the sweet analgesic is administered to the patient's intra-oral cavity 30 minutes before the procedure. In some embodiments, the sweet analgesic is sequentially administered or co-administered with one or more additional analgesics, a local or generic anesthetic, or other painkillers. Examples include but are not limited to articaine, novicaine, lidocaine, acetaminophen, demerol, aspirin, or ibuprofen.

The sweet analgesic may be administered to the patient once or multiple times as needed. For example, the sweet analgesic may be administered once prior to the procedure beginning or once after the procedure begins as a response to the patient's input. The sweet analgesic may be administered multiple times prior to, during, and after the procedure as necessary to increase comfort level and decrease pain response. In some embodiment, the sweet analgesic is not sufficient to achieve a comfort level for the patient but can be administered in combination with one or more one or more additional analgesics, a local or generic anesthetic, or other painkillers to reduce the amount of additional analgesic, anesthetic, or painkillers necessary for the patient to achieve a suitable comfort level during the procedure or treatment. In some embodiments, the sweet analgesic will be pre-moistened prior to administration to increase patient comfort. For example, a dental grade roll or pad containing the sweet analgesic can be moistened with sterile water to increase patient comfort during administration.

A kit comprising the sweet analgesic and a delivery vehicle for administration of the sweet analgesic to the intra-oral cavity of a patient prior to or during a medical procedure or treatment is disclosed. The kit may provide the sweet analgesic in sterile, individually wrapped packages. For example, a kit may provide dental grade rolls or pads presoaked with a sweetener individually wrapped for convenience of use. The kit may include a plurality of individually wrapped single doses. The kit may provide a container with multiple doses in a single dispenser. For example, if the sweet analgesic is delivered as a tablet, the kit can include a bottle containing 25, 50, 100, 200 or more tablets. The kit may include a bottle or toothpaste type container when the sweet analgesic is a gel. The toothpaste type container may be accompanied by a plurality of sterile applicators suitable for administration of the gel to the gum or cheek of the adult patient. The kit may include directions on how to administer the sweet analgesic with recommended doses, locations for administration within the oral cavity, safety instructions, ingredient information, side effects and the like.

### EXAMPLES

The present invention may be better understood with reference to the following examples. These examples are intended to be representative of specific embodiments of the invention, and are not intended as limiting the scope of the invention.

### Example 1

### Intra-Oral Administration of Sweet Analgesic During Dental Cleaning Increases the Comfort Level of Patients

Sixteen adult patients participated in the study to evaluate the comfort level of each patient during a dental hygiene visit. During one half of the cleaning, each patient was administered a routinely used dental roll (placebo). During the other half of the cleaning, each patient was administered a similar dental roll containing sucralose (sweetened roll). In each instance, the placebo or sweetened roll was placed between the lip and the gum of the patient for half of the cleaning procedure.

The sweetened dental rolls were prepared as follows. Sixty 1 gram packets of SPLENDA were dissolved in 150 mls of water. Fifty-five dental grade gauze rolls (Henry Schein rolls) were placed into the 150 milliliter mixture and allowed to absorb the entire mixture. Each dental roll absorbed approximately 2.5 mls of the mixture. The sweetened rolls were then dried on wax paper resulting in about 1 gram (1 packet) of SPLENDA per sweetened roll. One packet of SPLENDA contains 1.5% sucralose. Therefore, each sweetened roll contained about approximately 0.015 grams of sucralose, the balance of the SPLENDA comprising dextrin and maltodextrin. On average, the 16 patients were administered one sweetened roll during the cleaning procedure.

Following the cleaning, each patient evaluated how effective the sweetened roll was at improving the comfort of the dental cleaning. The patients could choose from one of the following six categories: (1) I couldn't tolerate the roll and asked that it be removed; (2) worse; (3) no difference between the rolls; (4) slightly better; (5) better; and (6) much better. Categories (1) and (2) are grouped together to represent patients who experienced a worse comfort level while using the sweetened roll. Category (3) represents patients who did not experience any difference between the rolls. Categories (4), (5), and (6) are grouped together to represent patients who experienced a better comfort level while using the sweetened roll. The results from the 16 patient study are shown in Table 2.

TABLE 2
Results of a 16 patient study identifying patient response
to using a sweetened roll during a dental cleaning.

| | Better Comfort | No | Worse Comfort |
|---|---|---|---|
| | Level | Difference | Level |
| Male | 6 | 2 | 0 |
| Female | 4 | 3 | 1 |
| Total | 10 | 5 | 1 |

63% of the patients experienced a better comfort level. 31% of the patients experienced no difference and only 6% of the patients experienced a worse comfort level.

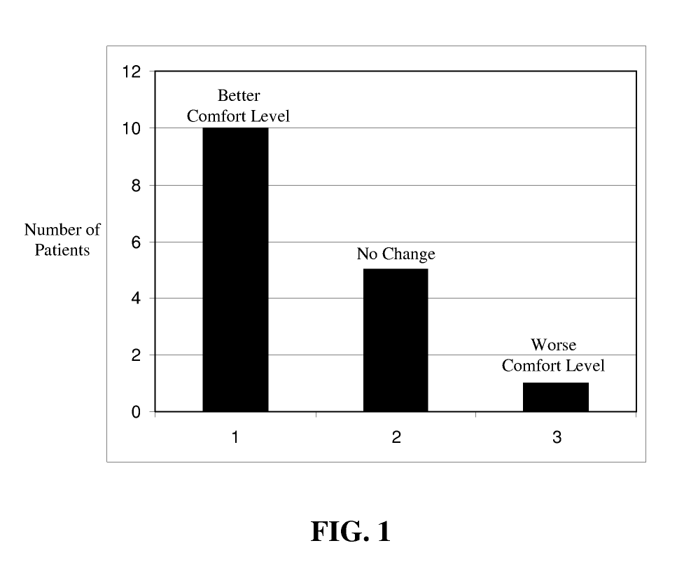

The results in Table 2 clearly demonstrate that using a sweetened roll increases the comfort level in a patient undergoing a dental cleaning in 63% of those tested. As shown in Table 2 and FIG. 1, ten patients (Group 1) experienced a better comfort level as a result of using the sweet roll during the dental cleaning. Five patients (Group 2) did not experience any change in comfort level as a result of using the sweet roll. Only one patient (Group 3) experienced a worse comfort level as a result of using the sweet roll. Remarkably, 75% of the men tested experienced a better comfort level while using the sweetened roll compared to only 50% of the women tested.

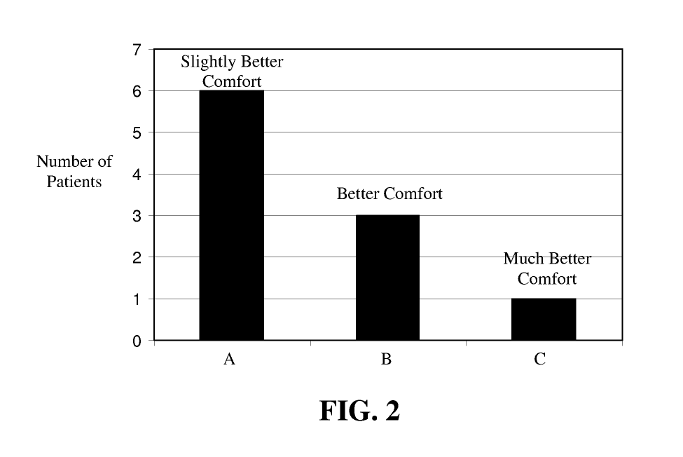

FIG. 1 also clearly shows that a sweet analgesic is effective at increasing patient comfort in adult patients during an intra-oral procedure. These findings were surprising and unexpected, and are contrary to the prevailing theory in the art which suggests the use of sucrose as an analgesic is limited to infants and neonates. It has previously been shown that analgesics may effectively reduce pain response in infants and neonates but recent studies had determined that sweet analgesics were not effective in patients other than infants. FIG. 2 shows the degree to which the sweet analgesic was capable of improving patient comfort during the dental procedure. Of the ten patients who reported a better comfort level, one patient (C) described the experience as much better, three patients (B) described the experience as better, and six patients (A) described the experience as slightly better. Without being limited to a particular theory, the continuous release of the sweetener or the high level of sweetness may account for the effectiveness of this invention.

Those skilled in the art will appreciate that the present invention may be embodied by forms that are not disclosed without departing from the spirit of fundamental attributes thereof. The description of the present invention discloses only some embodiments, a skilled artisan understands that other variations are contemplated as being with the scope of the present invention. Accordingly, the present invention is not limited in the particular embodiments which have been described in detail therein. Since many embodiments of the invention can be made without departing from the sprit and scope of the invention, the invention resides in the claims hereinafter appended.

What is claimed is:

1. A pain reliever composition for medical procedures or treatments comprising:
a sweet analgesic; and
a delivery vehicle, wherein the delivery vehicle is suitable for intra-oral delivery.

2. The pain reliever composition of claim 1, wherein the sweet analgesic comprises sucrose, glucose, fructose, dextrose, maltodextrin, corn syrup, high fructose corn syrup, cyclamate, aspartame, sucralose, xylitol, cyclamate, stevia, brazzein, curculin, erythritol, glycyrrhizin, honey, luo han gua, mabinlin, monatin, miraculin, monellin, pentadin, thaumatin, acesulfame potassium, alitame, salt of aspartame-acesulfame, dulcin, glucin, neohesperidin dihydrochalcone, neotame, P-4000, saccharin, or a combination thereof.

3. The pain reliever composition of claim 1, wherein the delivery vehicle comprises a capsule, pill, tablet, dental roll, dental pad, gauze, film, elixir, gel, gum, emulsion, syrup, suspension, ampoule, tincture, or combination thereof.

4. The pain reliever composition of claim 1, wherein the sweet analgesic comprises about 0.00001 grams to about 5.0 grams sucralose or a sweetening equivalent amount thereof.

5. The pain reliever composition of claim 1, wherein the medical procedure or treatment comprises a dental procedure or treatment, physical therapy procedure or treatment, or a treatment of chronic pain.

6. The pain reliever composition of claim 1, wherein the medical procedure or treatment comprises an intra-oral procedure.

7. The pain reliever composition of claim 6, wherein the intra-oral procedure comprises a dental cleaning, filling, tooth extraction, root canal, bleaching, the addition or removal of sealant, the addition or removal of orthodontics, the addition or removal of dentures, dental caps, or dental bridges.

8. A method of improving patient discomfort and/or pain associated with a medical procedure or treatment, comprising:
administering a sweet analgesic or a pain reliever composition according to claim 1 to the intra-oral cavity of a patient prior to or during the medical procedure or treatment

9. The method of claim 8, wherein the sweet analgesic comprises sucrose, glucose, fructose, dextrose, maltodextrin, corn syrup, high fructose corn syrup, cyclamate, aspartame, sucralose, xylitol, cyclamate, stevia, brazzein, curculin, erythritol, glycyrrhizin, honey, luo han gua, mabinlin, monatin, miraculin, monellin, pentadin, thaumatin, acesulfame potassium, alitame, salt of aspartame-acesulfame, dulcin, glucin, neohesperidin dihydrochalcone, neotame, P-4000, saccharin, or a combination thereof.

10. The method of claim 8, wherein the patient is an adult patient.

11. The method of claim 8, wherein the sweet analgesic is applied to a dental roll or pad.

12. The method of claim 11, wherein the dental roll or pad is placed between the cheek and gum of the patient's intra-oral cavity.

13. The method of claim 8, wherein the sweet analgesic comprises about 0.00001 grams to about 5.0 grams sucralose or a sweetening equivalent amount thereof.

14. The method of claim 8, wherein the medical procedure or treatment comprises a dental procedure or treatment, physical therapy procedure or treatment, or a treatment of chronic pain.

15. The method of claim 8, wherein the medical procedure or treatment comprises an intra-oral procedure.

16. The method of claim 15, wherein the intra-oral procedure comprises a dental cleaning, filling, tooth extraction, root canal, bleaching, the addition or removal of sealant, the addition or removal of orthodontics, the addition or removal of dentures, dental caps, or dental bridges.

17. A kit for improving patient discomfort and/or pain associated with a medical procedure or treatment, the kit comprising:
a sweet analgesic; and
a delivery vehicle, wherein the delivery vehicle is suitable for intra-oral delivery of the sweet analgesic.

18. The kit of claim 17, further comprising instructions for administering the sweet analgesic to the patient.

19. The kit claim 17, wherein the delivery vehicle comprises a capsule, pill, tablet, dental roll, dental pad, gauze, film, elixir, gel, gum, emulsion, syrup, suspension, ampoule, tincture, or combination thereof.

20. The kit of claim 19, wherein the delivery vehicle is a dental roll or pad.

21. The kit of claim 20, wherein the dental roll or pad comprises the sweet analgesic.

22. The kit of claim 17, wherein the delivery vehicle is individually wrapped.

23. The kit of claim 17, wherein the sweet analgesic comprises about 0.00001 grams to about 5.0 grams sucralose or a sweetening equivalent amount thereof.
